(19) United States
(12) Patent Application Publication
(10) Pub. No.: US 2024/0374187 A1
(43) Pub. Date: Nov. 14, 2024

(54) MULTI-MODAL SYSTEMS AND METHODS FOR VOICE-BASED MENTAL HEALTH ASSESSMENT WITH EMOTION STIMULATION

(71) Applicant: Wonder Technologies Pte. Ltd., Singapore (SG)

(72) Inventors: Biman Najika Liyanage, Beijing (CN); Zhengwen Zhu, Beijing (CN); Tai-ni Wu, Beijing (CN)

(21) Appl. No.: 18/783,405

(22) Filed: Jul. 24, 2024

Related U.S. Application Data
(63) Continuation of application No. PCT/IB2023/050566, filed on Jan. 24, 2023.

(30) Foreign Application Priority Data
Jan. 24, 2022 (SG) 10202200711S

(51) Int. Cl. A61B 5/16; A61B 5/00; G09B 5/04; G10L 15/02; G10L 25/18; G10L 25/63; G10L 25/90; G10L 25/93

(52) U.S. Cl. CPC A61B 5/165; A61B 5/4803; G09B 5/04; G10L 15/02; G10L 25/18; G10L 25/63; G10L 25/90; G10L 25/93

(57) ABSTRACT

Multi-modal systems, for voice-based mental health assessment with emotion stimulation, comprising: a task construction module to construct tasks for capturing acoustic, linguistic, and affective characteristics of speech of a user; a stimulus output module comprising stimuli, basis the constructed tasks, to be presented to a user in order to elicit a trigger of one or more types of user behaviour, the triggers being in the form on input responses; response intake module to present, to a user, the stimuli, and, in response, receive corresponding responses in one or more formats from responses; an autoencoder to define relationship/s, using the fused features, between: an audio modality to output extracted high-level text features; and a text modality to output extracted high-level audio features; the autoencoder to receive extracted high-level text and audio features, in parallel, to output a shared representation feature data set for emotion classification correlative to the mental health assessment.

### RELATED APPLICATIONS

This application claims priority under 35 U.S.C. § 120 to, and is a continuation of, co-pending International Application PCT/IB2023/050566, filed Jan. 24, 2023 and designating the US, which claims priority to SG Application 10202200711S, filed Jan. 24, 2022, such SG Application also being claimed priority to under 35 U.S.C. § 119. These SG and International applications are incorporated by reference herein in their entireties.

### FIELD

This invention relates to the field of artificial intelligence, machine learning, computational networks, and computer engineering. Particularly, this invention relates to multi-modal systems and methods for voice-based mental health assessment leveraging induced emotions and emotion recognition techniques.

### BACKGROUND

Mental health disorders are reported to be increasing at an accelerating rate affecting 10-15% world population catalyzed by the COVID-19 pandemic. The current rate of growth has a major economic impact compared to cancer, cardiovascular diseases, diabetes, and respiratory diseases. Suicide, owing to mental health issues, is currently the second leading cause of death in 15 to 29-year-olds, resulting in enormous social disruption and losses in productivity. In response to these alarming circumstances, in 2016, the World Health Organization declared depression to be the leading cause of disability worldwide.

However, as severity increases, traditional health care systems are not equipped to handle the massive influx of individuals affected by mental health disorders. Early detection and early intervention techniques can drastically improve cure rate of affected individuals; thus, relieving economic burden and increasing productivity of and quality of life of affected individuals. Automated screening and continuous monitoring have proven to be an effective alternative to facilitate screening.

Voice-based Artificial Intelligence (AI) based screening technologies are gaining popularity amount users detecting a variety of mental health diseases including depression and anxiety. It is deemed as an inexpensive scalable solution since it can be easily accessed through digital devices, for example, mobile phones. Leveraging voice as a “biomarker”, these technologies can also be more robust to dishonest or cheating behaviours during assessment or monitoring sessions rather than traditional outpatient screening mechanisms, for example, PHQ-9 (Patient Health Questionnaire 9) or GAD-7 (Generalized Anxiety Disorder 7).

Voice-based Artificial Intelligent (AI) screening systems and methods, usually, rely on scripted dialogs and collect audio-based answers (other signals might also be collected at the same time). Based on the answers, it may use models built on acoustic features, or acoustic plus NLP features to produce a final classification; this is well documented in prior art. In order to avoid taking not useful answers as input, certain statistics-based techniques might be used to test each answer before moving to next step. A simple solution is to measure the total time of the recorded answer, and a more complex one involves something like voice activation or leveraging ASR (Automatic Speech Recognition) to check whether there are sentences with enough words. If the preconfigured threshold is not met, user might get re-prompted with the same question (or a different one) and will have to answer again.

### SUMMARY

There are pitfalls with this kind of approach of the prior art:

First, it gives longer answers a natural bias which is not always correct. A relatively short answer might reflect truthfully a user's mental health status, however a longer one, with more words, on the other hand, might lead to incorrect assessment when the user is dishonest.

Second, the ASR (Automatic Speech Recognition) technology used, in such system and methods, usually ignores certain speech behaviours, e.g. pauses, hesitations, jittering, and shimmering etc., and only produces the sentences/words it can recognize. Filtering, based on output from such ASR technology might, leave out some otherwise meaningful answers to screening models.

Finally, a user's emotion status when answering a question might influence a mental health assessment result. Answer validation techniques, of the prior art, however, ignores emotion, as an input, and is not sufficient here either.

Therefore, a better mechanism is needed to help ensure relatively higher input quality, which will, in turn, improve model performance.

Furthermore, in recent years, non-invasive and continuous monitoring of physiological and psychological data by smartphone-based technologies and wearable devices has aroused researchers' interests. Advancements in acoustic and speech processing makes a new frontier of behavioral health diagnosis using machine learning. It was found that the depressive patients showed reduced stress, mono-pitch, loudness decay and etc. in language, which was consistent with the clinical observations of depressed patients. These patients showed slower speech, more pauses, and low volume compared to ordinary people. Studies have shown that the speech biomarkers recognized by machine learning models have a positive correlation and have greater potential in detecting mental disorders such as depression. Researchers have spent tremendous amount of effort studying depression and its correlation to acoustics and semantic aspects of speech.

AI technologies, in this space, can be divided into three main categories:
1. Semantic based: Transform speech data into text transcriptions by applying automatic speech recognition (ASR), then perform natural language process (NLP) on top to build natural language-based classification models.
2. Acoustic based: Extract acoustic features from speech directly, and then build classification models based on them. These features can be either hand engineered features e.g. rhythmic features/spectral-based correlation features, or latent feature embeddings via pretrained models.
3. Multi-modal based: There are also some tries in combining these two modalities to create a multi-modal AI model to increase the accuracy of the assessment.

These technologies and related studies often use voice recordings from users related to certain topic as the model input; for example, answers to certain fixed questions about their rest schedule, or description of their health status, and the like. On the other hand, it is worth noting that major depressive disorder (MDD) usually involves the numbing of emotions, especially grief, fear, anger and shame. Many past reviews suggest that individuals with depression differ from non-depressed individuals, especially in their emotion preferences and use of emotion regulation strategies. Unfortunately, users' emotion preference or emotion regulation capabilities are not paid enough attention in existing voice-based AI technologies from training data collection, to model training, to inferencing when model is applied in real applications.

It has become a known fact, based on previous research, that human voice conveys a lot of emotion information. For example, some prior art literatures have found that one can detect not just basic emotional tone in the voice (e.g., positive vs. negative feelings or excitement vs. calm) but fine emotional nuances. Covering more emotion effects actively or utilizing emotion information better from voice (especially valence) can potentially the accuracy of such AI models.

This patent discusses a system and method towards building such technology.

An object of the invention is to assess mental health based on voice-based biomarkers in conjunction with emotion determination/recognition.

Another object of the invention is to eliminate biases in assessing mental health based on voice-based biomarkers leveraging induced emotions as well as emotion recognition.

Yet another object of the invention is to provide an enhanced filtering technique whilst assessing mental health based on voice-based biomarkers.

Still another object of the invention is to ensure relatively higher input quality, of voice signals, whilst assessing mental health based on voice-based biomarkers.

An additional another object of the invention is to enhance speech behaviour understanding whilst assessing mental health based on voice-based biomarkers.

According to this invention, there are provided multi-modal systems and methods for voice-based mental health assessment with emotion stimulation.

According to this invention, there are provided multi-modal systems for voice-based mental health assessment with emotion stimulation, the system comprising:
a task construction module configured to construct tasks for capturing acoustic, linguistic, and affective characteristics of speech of a user;
a stimulus output module configured to receive data from the task construction module, the stimulus output module comprising one or more stimuli, basis the constructed tasks, to be presented to a user in order to elicit a trigger of one or more types of user behaviour, the triggers being in the form on input responses;
response intake module configured to present, to a user, the one of more stimuli, basis the constructed tasks, from the stimulus output module, and, in response, receive corresponding responses in one or more formats;
a features' module comprising:
   a feature constructor configured to define features, for each of the constructed task, the features being defined in terms of learnable heuristic weighted tasks;
   a feature extractor configured to extract one or more defined features from the receive corresponding responses correlative to the constructed tasks, with learnable heuristic weighted model considering at least one selected from a group consisting of ranked constructed tasks;
   a feature fusion module configured to fuse two or more defined features in order to obtain fused features;
an autoencoder to define relationship/s, using the fused features, between:
   an audio modality, of the feature fusion module, working in consonance with the response intake module extract high-level features, in the responses, so as to output extracted high-level text features; and
   a text modality, of the feature fusion module, working in consonance with the response intake module to extract high-level features, in the responses, so as to output extracted high-level audio features;
said autoencoder configured to receive extracted high-level text features and extracted high-level audio features, in parallel, from the audio modality and the text modality to output a shared representation feature data set for emotion classification correlative to the mental health assessment.

In at least an embodiment of the system, the constructed tasks being articulation tasks and/or written tasks.

In at least an embodiment of the system, the task construction module comprises a first order ranking module configured to rank the constructed tasks in order of difficulty; thereby, assigning a first order of weights to each constructed task.

In at least an embodiment of the system, the task construction module comprises a first order ranking module configured to rank the constructed tasks in order of difficulty; thereby, assigning a first order of weights to each constructed task, in that, the construed tasks being stimuli marked with a ranked valence level, correlative to analysed responses, selected from a group consisting of positive valence, negative valence, and neutral valence.

In at least an embodiment of the system, the task construction module comprises a second order ranking module configured to rank complexity of the constructed tasks in order of complexity; thereby, assigning a second order of weights to each constructed task.

In at least an embodiment of the system, the task construction module comprises a second order ranking module configured to rank complexity of constructed tasks and affective expectation in terms of response vectors, of the ranked constructed tasks in order to create a data collection pool.

In at least an embodiment of the system, the constructed tasks being selected from a group of tasks consisting of cognitive tasks of counting numbers for a pre-determined time duration, tasks correlating to pronouncing vowels for a pre-determined time duration, uttering words with voiced and unvoiced components for a pre-determined time duration, word reading tasks for a pre-determined time duration, paragraph reading tasks for a pre-determined time duration, tasks related to reading paragraphs with phoneme and affective complexity to open-ended questions with affective variation, and pre-determined open tasks for a for a pre-determined time duration.

In at least an embodiment of the system, the constructed tasks comprising one or more questions, as stimulus, each question being assigned a question embedding with a 0-N vector such that a question-specific feature extractor is trained in relation to determination of embeddings' extraction, from the question, correlative to word-embedding, phone-embedding, and syllabl-level embedding, the extracted embeddings being and forced aligned for a mid-level feature fusion.

In at least an embodiment of the system, the one or more stimulus being selected from a group of stimuli consisting of audio stimulus, video stimulus, combination of audio and video stimulus, text stimulus, multimedia stimulus, physiological stimulus, and its combinations.

In at least an embodiment of the system, the one or more stimulus comprising stimulus vectors calibrated to elicit textual response vectors and/or audio response vectors and/or video response vectors and/or multimedia response vectors and/or physiological response vectors in response to the stimulus vectors.

In at least an embodiment of the system, the one or more stimulus being parsed through a first vector engine configured to determine its constituent vectors in order to determine a weighted base state in correlation to such stimulus vectors.

In at least an embodiment of the system, the one or more responses being selected from a group of responses consisting of audio responses, video responses, combination of audio and video responses, text responses, multimedia responses, physiological responses, and its combinations.

In at least an embodiment of the system, the one or more stimulus comprising response vectors correlative to elicited audio response vectors and/or elicited video response vectors in response to stimulus vectors of the stimulus output module.

In at least an embodiment of the system, the one or more stimulus being parsed through a first vector engine configured to determine its constituent vectors in order to determine a weighted base state in correlation to such stimulus vectors.

In at least an embodiment of the system, the response intake module comprising a passage reading module configured to allow users to perform tasks correlative to reading passages for a pre-determine time.

In at least an embodiment of the system, the feature constructor being a Geneva Minimalistic Acoustic Parameter Set (GeMAPS) based feature constructor for analysis of audio responses.

In at least an embodiment of the system, the feature constructor being a Geneva Minimalistic Acoustic Parameter Set (GeMAPS) based feature constructor, the feature constructor:
using a set of 62 parameters to analyse speech;
providing a symmetric moving average filter, 3 frames long, to smooth over time, the smoothing being performed within voiced regions, of the responses, for pitch, jitter, and shimmer;
applying arithmetic mean and coefficient of variation as functionals to 18 low-level descriptors (LLDs), yielding 36 parameters;
applying 8 functions to loudness;
applying 8 functions to pitch;
determining arithmetic mean of Alpha Ratio;
determining Hammarberg Index;
determining spectral features vide spectral slopes from 0-500 Hz and 500-1500 Hz over all unvoiced segments;
determining temporal features of continuously voiced and unvoiced regions from the responses; and
determining Viterbi-based smoothing of a F0 contour, thereby, preventing single voiced frames which are missing by error.

In at least an embodiment of the system, the feature constructor being a Geneva Minimalistic Acoustic Parameter Set (GeMAPS) based feature constructor, the feature constructor being configured with a set of low-level descriptors (LLDs) for analysis of the spectral, pitch, and temporal properties of the responses being audio responses, the features being selected from a group of features consisting of:
Mel-Frequency Cepstral Coefficients (MFCCs) and their first and second derivatives;
Pitch and pitch variability;
Energy and energy entropy;
Spectral centroid, spread, and flatness;
Spectral slope;
Spectral roll-off;
Spectral variation;
Zero-crossing rate;
Shimmer, jitter; and harmonic-to-noise ratio;
Voice-probability (based on pitch); and
Temporal features like the rate of loudness peaks, and the mean length and standard deviation of continuously voiced and unvoiced regions

In at least an embodiment of the system, the feature constructor being a Geneva Minimalistic Acoustic Parameter Set (GeMAPS) based feature constructor, the feature constructor being configured with a set of frequency related parameters selected from a group of frequency-related parameters consisting of:
Pitch, logarithmic F0 on a semitone frequency scale, starting at 27.5 Hz (semitone 0);
Jitter, deviations in individual consecutive F0 period lengths;
Formant 1, 2, and 3 frequency, centre frequency of first, second, and third formant;
Formant 1, bandwidth of first formant;
Energy related parameters;
Amplitude related parameters;
Shimmer, difference of the peak amplitudes of consecutive F0 periods;
Loudness, estimate of perceived signal intensity from an auditory spectrum;
Harmonics-to-Noise Ratio (HNR), relation of energy in harmonic components to energy in noiselike components;
Spectral (balance) parameters;
Alpha Ratio, ratio of the summed energy from 50-1000 Hz and 1-5 kHz;
Hammarberg Index, ratio of the strongest energy peak in the 0-2 kHz region to the strongest peak in the 2-5 kHz region;
Spectral Slope 0-500 Hz and 500-1500 Hz, linear regression slope of the logarithmic power spectrum within the two given bands;
Formant 1, 2, and 3 relative energy, as well as the ratio of the energy of the spectral harmonic peak at the first, second, third formant's centre frequency to the energy of the spectral peak at F0;
Harmonic difference H1-H2, ratio of energy of the first F0 harmonic (H1) to the energy of the second F0 harmonic (H2); and
Harmonic difference H1-A3, ratio of energy of the first F0 harmonic (H1) to the energy of the highest harmonic in the third formant range (A3).

In at least an embodiment of the system, the feature constructor being a Geneva Minimalistic Acoustic Parameter Set (GeMAPS) based feature constructor, the feature constructor using Higher order spectra (HOSA) functions, the functions being functions of two or more component frequencies achieving bispectrum frequencies.

In at least an embodiment of the system, the feature constructor being a Geneva Minimalistic Acoustic Parameter Set (GeMAPS) based feature constructor, the feature constructor using Higher order spectra (HOSA) functions, the functions being functions of two or more component frequencies achieving bispectrum frequencies, in that, the bispectrum using third-order cumulants to analyze relation between frequency components in a signal correlative to the responses for examining nonlinear signals.

In at least an embodiment of the system, the feature extractor fuses higher-level feature embeddings using mid-level fusion.

In at least an embodiment of the system, the feature extractor comprising a dedicated linguistic feature extractor, with learnable weights per stimulus, correlative to linguistic tasks.

In at least an embodiment of the system, the feature extractor comprising a dedicated affective feature extractor, with learnable weights per stimulus, correlative to affective tasks.

In at least an embodiment of the system, the feature fusion module comprising an audio module configured with high-level feature extractors and at least an autoencoder-based feature fusion to classify emotions from one or more responses.

In at least an embodiment of the system, the feature fusion module comprising an audio module configured with high-level feature extractors and at least an autoencoder-based feature fusion to classify emotions from one or more responses, in that, the audio modality using a local question-specific feature extractor to extract high-level features from a time-frequency domain relationship in the responses so as to output extracted high-level audio features.

In at least an embodiment of the system, the feature fusion module comprising a text module configured with high-level feature extractors and at least an autoencoder-based feature fusion to classify emotions from one or more responses.

In at least an embodiment of the system, the feature fusion module comprising a text module configured with high-level feature extractors and at least an autoencoder-based feature fusion to classify emotions from one or more responses, in that, the text modality using a Bidirectional Long Short-Term Memory network with an attention mechanism to simulate an intra-modal dynamic so as to output extracted high-level text features.

In at least an embodiment of the system, the feature fusion module comprising an audio module:
using extracted features in acoustic feature embeddings from pre-trained models;
comparing the extracted features on a spectral domain;
determining vocal tract co-ordination features;
determining recurrent quantification analysis features;
determining Bigram count features and bigram duration features corerelative to speech landmarks; and
fusing the features in an autoencoder.

In at least an embodiment of the system, the feature extractor comprising a speech landmark extractor configured to determine event markers associated with the responses, the determination of the event markers being correlative to location of acoustic events, from the response, in time, the determination including determination of timestamp boundaries that denote sharp changes in acoustic responses, independently of frames.

In at least an embodiment of the system, the feature extractor comprising a speech landmark extractor configured to determine event markers associated with the responses, each of the event markers having onset values and offset values, the event markers being selected from a group of landmarks consisting of glottis-based landmark (g), periodicity-based landmark (p), sonorant-based landmark(s), fricative-based landmark (f), voiced fricative-based landmark (v), and bursts-based landmark (v), each of the landmark being used to determine points in time, at which different abrupt articulatory events occur, correlative to rapid changes in power across multiple frequency ranges and multiple time scales.

In at least an embodiment of the system, the autoencoder being configured with a multi-modal multi-question input fusion architecture comprising:
at least an encoder for mapping one or more the characteristics with a task type to a lower-dimensional representation, each task being multiplied by the characteristics with a learnable weight-encoded matrix based on task type, the weights being correlative to mental health assessment;
at least a decoder for mapping the lower-dimensional representation to the one or more the characteristics in order to output mental health assessment; and
said autoencoder being trained to minimize reconstruction error between input tasks and decoder output by using a loss function.

According to this invention, there are provided multi-modal methods for voice-based mental health assessment with emotion stimulation, the method comprising the steps of:
constructing tasks for capturing acoustic, linguistic, and affective characteristics of speech of a user;
receiving data the constructed tasks, comprising one or more stimuli, basis the constructed tasks, to be presented to a user in order to elicit a trigger of one or more types of user behaviour, the triggers being in the form on input responses;
presenting, to a user, the one of more stimuli, basis the constructed tasks, and, in response, receiving corresponding responses in one or more formats;
defining features, for each of the constructed task, the features being defined in terms of learnable heuristic weighted tasks;
extracting one or more defined features from the receive corresponding responses correlative to the constructed tasks, with learnable heuristic weighted model considering at least one selected from a group consisting of ranked constructed tasks;
fusing two or more defined features in order to obtain fused features;
defining relationship/s, using the fused features, between:
   an audio modality, of the feature fusion module, working in consonance with the responses in order to extract high-level features, in the responses, so as to output extracted high-level text features; and
   a text modality, of the feature fusion module, working in consonance with the responses in order to extract high-level features, in the responses, so as to output extracted high-level audio features;
   said step of defining relationship/s comprising a step of receiving extracted high-level text features and extracted high-audio text features, in parallel, from the audio modality and the text modality to output a shared representation feature data set for emotion classification correlative to the mental health assessment.

In at least an embodiment of the method, the constructed tasks comprising one or more questions, as stimulus, each question being assigned a question embedding with a 0-N vector such that a question-specific feature extractor is trained in relation to determination of embeddings' extraction, from the question, correlative to word-embedding, phone-embedding, and syllabl-level embedding, the extracted embeddings being and forced aligned for a mid-level feature fusion.

In at least an embodiment of the method, the step of defining relationship/s comprising a step of configuring a multi-modal multi-question input fusion architecture comprising:
mapping, via an encoder, one or more the characteristics with a task type to a lower-dimensional representation, each task being multiplied by the characteristics with a learnable weight-encoded matrix based on task type, the weights being correlative to mental health assessment;
mapping, via a decoder, the lower-dimensional representation to the one or more the characteristics in order to output mental health assessment;
training to minimize reconstruction error between input tasks and decoder output by using a loss function.

In at least an embodiment of the method, the step of extracting high-level audio features comprising a step of extracting high-level features from a time-frequency domain relationship in the responses so as to output extracted high-level audio features.

In at least an embodiment of the method, the step of extracting high-level text features comprising a step of using a Bidirectional Long Short-Term Memory network with an attention mechanism to simulate an intra-modal dynamic so as to output extracted high-level text features.

### BRIEF DESCRIPTION OF THE ACCOMPANYING DRAWINGS

The patent or application file contains at least one drawing executed in color. Copies of this patent or patent application publication with color drawing(s) will be provided by the Office upon request and payment of the necessary fee.

The invention will now be described in relation to the accompanying drawings, in which:

FIG. 1 illustrates a schematic block diagram of a computing environment;

FIG. 2 illustrates a system for Training Data Collection with Induced Emotion;

FIG. 3 illustrates a sample induced emotion question set showing some tasks/questions in order to elicit emotion-based responses;

FIG. 4 illustrates known interplay between components, of speech, forming useful speech criteria;

FIG. 5 illustrates one such fixed reading passage;

FIG. 6 illustrates a phenome map, basis read passage by a user, without tones;

FIG. 7 illustrates another such fixed reading passage;

FIG. 8 illustrates a graph of phenomes versus occurrence, basis read passage by a user;

FIG. 9 illustrates representations of HOSA functions with respect to various mental states of a user;

FIG. 10 illustrates autocorrelations and cross-correlations between the 1st and the 2nd delta MFCCs extracted from a 10-s speech file, framework for extracting the delayed correlations from acoustic files reflecting the psychomotor retardation;

FIG. 11 illustrates a schematic block diagram of an autoencoder;

FIG. 12A to 12H illustrate various graphs, for at least one type of corresponding task (question), according to a non-limiting exemplary embodiment, with its original label being selected from ‘healthy’ or ‘depressed’, the graph showing phonemes and segments that have correlations with the model prediction, the areas highlight the parts of speech file that have features with high activation; for various users at various start times.

FIG. 13 illustrates a flowchart; and

FIG. 14 illustrates the high-level flowchart for voice-based mental health assessment with emotion stimulation.

### DETAILED DESCRIPTION

According to this invention, there are provided multi-modal systems and methods for voice-based mental health assessment with emotion stimulation. Mental health issues are tightly coupled with emotions. When deciding whether a user input is useful to the system, user's emotion is at least as important as the length of the answer, if not more.

The present disclosure may be a system, a method, and/or a computer program product, and/or a mobile device program/product. The computer program product may include a computer readable storage medium (or media) having computer readable program instructions thereon for causing a processor to carry out aspects of the present disclosure.

Aspects of the disclosed embodiments may include tangible computer readable media that store software instructions that, when executed by one or more processors, are configured for and capable of performing and executing one or more of the methods, operations, and the like consistent with the disclosed embodiments. Also, aspects of the disclosed embodiments may be performed by one or more processors that are configured as special-purpose processor(s) based on software instructions that are programmed with logic and instructions that perform, when executed, one or more operations consistent with the disclosed embodiments.

In describing the invention, the following definitions are applicable throughout (including above).

A “computer” may refer to one or more apparatus or one or more systems that are capable of accepting a structured input, processing the structured input according to prescribed rules, and producing results of the processing as output. Examples of a computer may include: a computer; a stationary or portable computer; a computer having a single processor, multiple processors, or multi-core processors, which may operate in parallel or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer or software, such as, for example, a digital signal processor (DSP), a field-programmable gate array (FPGA), an application specific integrated circuit (ASIC), an application specific instruction-set processor (ASIP), a chip, chips, or a chip set; a system-on-chip (SoC) or a multiprocessor system-on-chip (MPSoC); an optical computer; a quantum computer; a biological computer; and an apparatus that may accept data, may process data in accordance with one or more stored software programs, may generate results, and typically may include input, output, storage, arithmetic, logic, and control units.

“Software” may refer to prescribed rules to operate a computer or a portion of a computer. Examples of software may include: code segments; instructions; applets; pre-compiled code; compiled code; interpreted code; computer programs; and programmed logic.

A “computer-readable medium” may refer to any storage device used for storing data accessible by a computer. Examples of a computer-readable medium may include: a magnetic hard disk; a floppy disk; an optical disk, such as a CD-ROM and a DVD; a magnetic tape; a memory chip; or other types of media that can store machine-readable instructions thereon.

A “computer system” may refer to a system having one or more computers, where each computer may include computer-readable medium embodying software to operate the computer. Examples of a computer system may include: a distributed computer system for processing information via computer systems linked by a network; two or more computer systems connected together via a network for transmitting or receiving information between the computer systems; and one or more apparatuses or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those that may be made through telephone or other communication links. A network may further include hard-wired connections (e.g., coaxial cable, twisted pair, optical fiber, waveguides, etc.) or wireless connections (e.g., radio frequency waveforms, free-space optical waveforms, acoustic waveforms, satellite transmissions, etc.). Examples of a network may include: an internet, such as the Internet; an intranet; a local area network (LAN); a wide area network (WAN); and a combination of networks, such as an internet and an intranet. Exemplary networks may operate with any of a number of protocols, such as Internet protocol (IP), asynchronous transfer mode (ATM), or synchronous optical network (SONET), user datagram protocol (UDP), IEEE 802.x, etc.

The terms “data” and “data item” as used herein refer to sequences of bits. Thus, a data item may be the contents of a file, a portion of a file, a page in memory, an object in an object-oriented program, a digital message, a digital scanned image, a part of a video or audio signal, or any other entity which can be represented by a sequence of bits. The term “data processing” herein refers to the processing of data items, and is sometimes dependent on the type of data item being processed. For example, a data processor for a digital image may differ from a data processor for an audio signal.

The term “first”, “second”, and the like, herein do not denote any order, preference quantity or importance, but rather are used to distinguish one element from another, and the terms “a” and “an” hereinafter do not denote a limitation of quantity, but rather denote the presence of at least one of the referenced items.

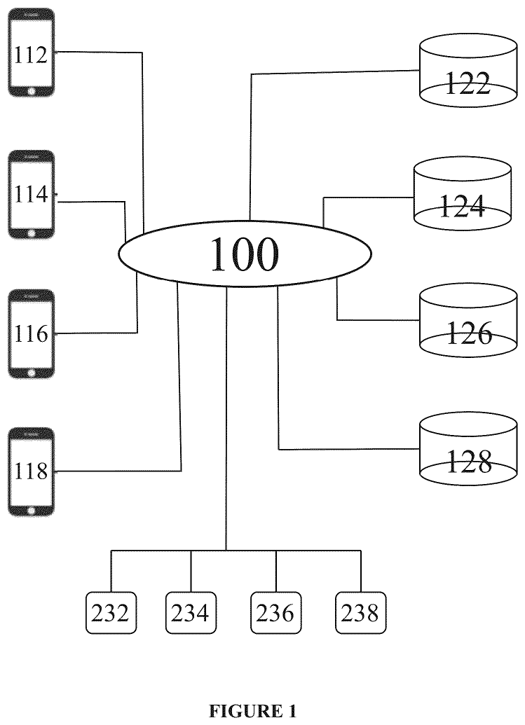

FIG. 1 illustrates a schematic block diagram of a computing environment comprising a networked server (100) and one or more networked client devices (112, 114, 116, 118) interfacing with the networked server (100), by means of a network, and one or more databases (122, 124, 126, 128) interfacing with the networked server (100).

Spoken language may seem effortless, but it is actually a complex process that involves the coordination of cognitive and physiological actions in the brain. It requires a series of intricate movements to be executed effectively. Normal speech originates from a body's physiological processes, starting with creation of potential energy in lungs and changes in air pressure within vocal tract. When speaking with a voiced sound, the lungs release air and speed of this air affects regularity of the vocal folds. As a harmonically rich sound energy from the glottis travels through the vocal tract and laryngeal flow, amplitudes of the harmonics are modified by pharyngeal, oral, and nasal cavities, as well as by movements of articulators (such as the tongue, teeth, lips, jaw, and velum) acting as filters.

Depression and psychomotor retardation are associated with impaired vocal function and weaker harmonic formant amplitudes and other abnormalities in some depressed individuals. This can also lead to a perceived “breathiness” in their speech, due to lack of laryngeal control caused by psychomotor retardation. This is in contrast to speech of healthy individuals. Intensity has also been shown to be a strong indicator of depression severity. Patients with depression may often have a weak vocal intensity and appear to speak with a monotone voice.

Researchers have studied how people with depression use language, both, (a) through listening to recordings of their speech, and (b) analyzing written text.

People with depression often struggle with verbal language skills, such as using inappropriate or unclear words, leaving sentences unfinished, and repeating themselves excessively.

The acoustic prosodic elements that are vital for identifying depression disorders may be abnormal, and these elements also interact with linguistic information (such as words and phrase meaning) to express an individual's emotional state.

In the past, subjective assessments of depressed individuals often focused on their speech patterns and related behaviors, based on preconceived notions of how people with depression should behave emotionally.

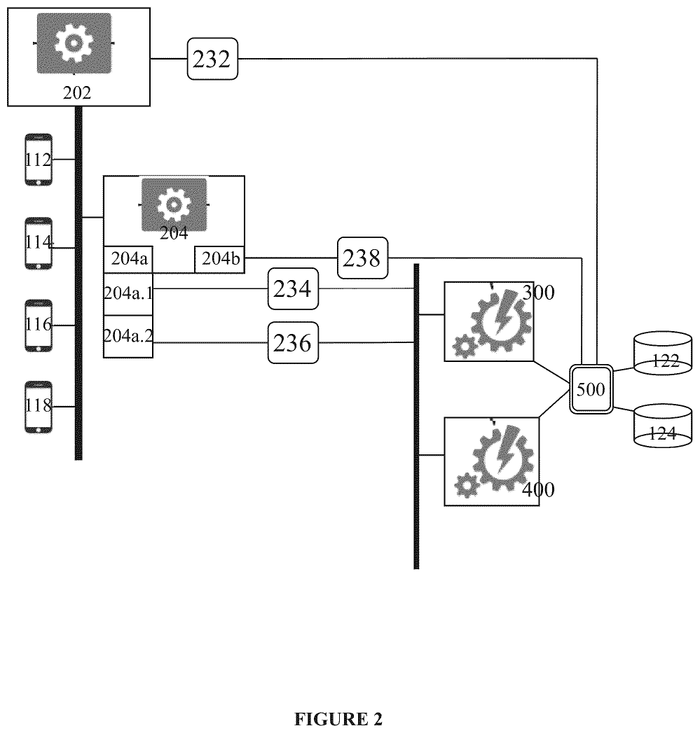

FIG. 2 illustrates a system for Training Data Collection with Induced Emotion.

In at least an embodiment, client devices (112, 114, 116, 118) are communicably coupled with at least a stimulus output module (202) configured to render one or more stimuli to a user of such client devices.

In at least an embodiment, the stimulus output module (202), is configured to provide an output stimulus to a user in order to elicit user response in the form of input responses. The output stimulus may comprise audio stimulus, video stimulus, combination of audio and video stimulus, text stimulus, multimedia stimulus, physiological stimulus, its combinations, and the like stimuli. The stimulus may comprise stimulus vectors calibrated to elicit textual response vectors and/or audio response vectors and/or video response vectors and/or multimedia response vectors and/or physiological response vectors in response to the stimulus vectors. One or more of the output stimuli is parsed through a first vector engine (232) configured to determine its constituent vectors in order to determine a weighted base state in correlation to such stimulus vectors.

In some embodiments, the stimulus output module (202) is used to send voice-based tasks to target subjects (users) with a given mental health disease (based on certain criteria e.g. DSM-V diagnosis) as well as to healthy subjects (users) with no mental health disease. This is done via the networked server (100) and through the users'/subjects' client devices (112, 114, 116, 118). In preferred embodiments, these stimuli are vector-configured stimuli to induce certain emotion reactions in subjects, these emotion reactions being, but not limited to:
happiness emotion reaction;
sadness emotion reaction;
neutral emotion reaction.

In some embodiments, the stimulus output module (202) comprises stimuli to trigger certain user/subject behaviours; e.g. imperative questions forcing user to repeat fixed number of words or vowels.

Embodiments of the present disclosure may include a task construction module configured to construct tasks for capturing acoustic, linguistic, and affective characteristics of speech of a user. Data, from the task construction module, is provided to the stimulus output module (202). Preferably, these tasks are articulation tasks since this invention deals with voice-based biomarkers.

In at least an embodiment, the task construction module comprises a first order ranking module configured to rank the constructed tasks in order of difficulty; thus, assigning weights to each task. In preferred embodiment, in order to better capture a user's emotion preferences and use of emotion regulation strategies related to mental, it is necessary to actively stimulate the user (speaker) for different valence (ranked) levels (positive, negative and neutral) during recording of their responses.

In at least an embodiment, the task construction module comprises a second order ranking module configured to rank complexity of questions and affective expectation of the ranked questions in order to create a data collection pool.

In multi-class classification, depression levels have an ordinal relationship; therefore, articulation tasks, from the task construction module, for different depression levels, also have learnable weights in order to optimize loss per question as well as per depression level.

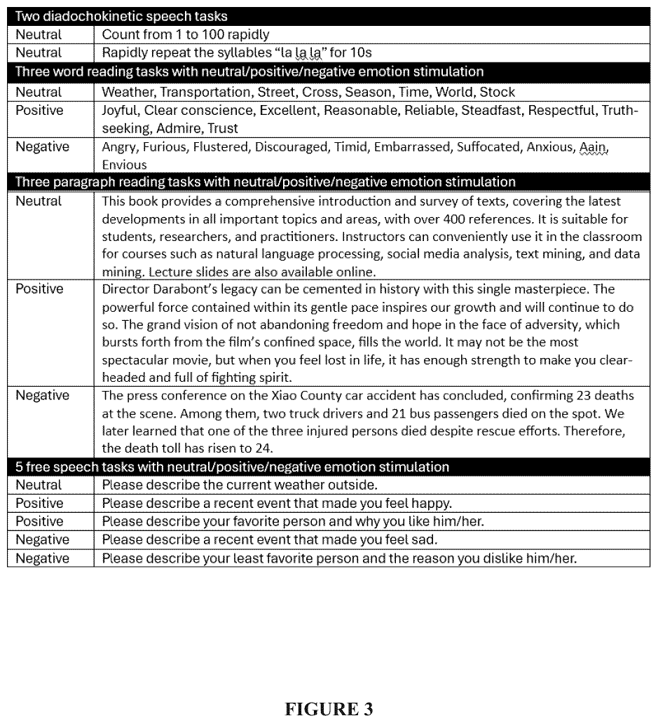

FIG. 3 illustrates a sample induced emotion task set (question set) showing some voice tasks in order to elicit emotion-based responses. Below is the emotion eliciting patterns for each voice task in at least an embodiment. Well design tasks not only cover different emotions well, but also have good coverage at phoneme level for the target language.

TABLE 1

| | | Induced | | |
|---|---|---|---|---|
| Task | Description | Emotion | Requirement | Example |
| 1 | Number | Neutral | 10-60 seconds | |
| | counting | | | |
| 2 | Pronouncing “la” | Neutral | 10-60 seconds | |
| | continuously | | | |
| 3 | Word reading | Neutral | 10-60 seconds | 8 words |
| 4 | Word reading | Positive | 10-60 seconds | 10 positive |
| | | | 8 positive words | words |
| 5 | Word reading | Negative | 10-60 seconds | 10 negative |
| | | | 8 negative words | words |
| 6 | Paragraph | Neutral | 10-60 seconds | |
| | reading | | | |
| 7 | Paragraph | Positive | 10-60 seconds | Paragraph |
| | reading | | Minimum 8 | with 17 |
| | | | positive | positive |
| | | | words, >20% | words |
| | | | are positive | |
| 8 | Paragraph | Negative | 10-60 seconds | Paragraph |
| | reading | | Minimum 8 | with 9 |
| | | | negative | negative |
| | | | words, >20% | words |
| | | | are negative | |
| 9 | Open task | Neutral - | 10-60 seconds | |
| | | weather | | |
| 10 | Open task | Positive - | 10-60 seconds | |
| | | Happy | | |
| | | experience | | |
| 11 | Open task | Positive - | 10-60 seconds | |
| | | People you | | |
| | | like | | |
| 12 | Open task | Negative - | 10-60 seconds | |
| | | Sad | | |
| | | experience | | |
| 13 | Open task | Negative - | 10-60 seconds | |
| | | People you | | |
| | | dislike | | |

In at least an embodiment, client devices (112, 114, 116, 118) are communicably coupled with at least a response intake module (204) configured to present, to a user, the one or more stimuli, from the stimulus output module (202), to a user of such client devices and, in response, receive corresponding inputs in one or more formats.

In at least an embodiment of the response intake module (204), there is provided an intake module (204a) configured to capture a user's input responses, in response to the output stimulus. The input responses may comprise audio inputs, video inputs, combination of audio and video inputs, multimedia inputs, and the like inputs. The input responses may comprise response vectors correlative to the elicited audio response vectors and/or elicited video response vectors in response to the stimulus vectors of the stimulus output module (202).

Embodiments of the present disclosure may include a data preprocessing module. In some embodiments of the task construction module, question construction may comprise construction questions from simple cognitive tasks of counting numbers, pronouncing vowels, uttering words with voiced and unvoiced components, reading paragraphs with phoneme and affective complexity to open-ended questions with affective variation and cognitive sentences generation and reaction may be collected through the response intake module (204).

Embodiments of the intake module (204a) may also include constructing and evaluating the responses, record answers, processing the response vectors till the boundary conditions may be met as a qualified data point for training. Response vectors may also include flash cards for data collection specific tasks, collecting metadata on user recordings also with task completion and qualification details.

Embodiments of the response intake module (204) may include a first measurement module configured to analyse, measure, and output, as a set of first measurement data, at least the following:
measured content understanding with respect to the response vectors;
measured response correctness with respect to the response vectors;
measured acoustic signal quality with respect to the response vectors.

Embodiments of the response intake module (204) may include a second measurement module configured to analyse, measure, and output, as a set of second measurement data, at least the following:
measured silence portions, from the response vectors;
measured signal-to-noise ratio, from the response vectors;
measured speech clarify, from the response vectors;
measured liveliness quotient, from the response vectors.
Based on pre-set thresholds, of the aforementioned second measurement data.

In preferred embodiments, segments, of response vectors, having measured signal-to-noise ratio greater than 15 are used as qualified speech samples by the system and method of this invention.

In at least an embodiment of the intake module (204a), there is provided at least an acoustic intake module (204a.1) configured to capture a user's acoustic input responses, in response, the one or more output stimuli, in the form of response acoustic signals. The response acoustic signals comprise response acoustic vectors which are correlated with the stimulus vectors of the stimulus output module (202) by a second vector engine (234) which determines constituent response acoustic vectors in order to determine a first state, of a user, in correlation to such stimulus vectors of the stimulus output of the stimulus output module (202).

In at least an embodiment of the intake module (204a), there is provided at least a textual intake module (204a.2), communicably coupled to the acoustic intake module (204a.1), in order to transcribe, via a transcription engine, the user's acoustic input responses, obtained from the acoustic intake module (204a.1), in order to provide response textual signals. The response textual signals comprise response textual vectors which are correlated with the stimulus vectors of the stimulus output module (202) by a third vector engine (236) which determines constituent response textual vectors in order to determine a second state, of a user, in correlation to such stimulus vectors of the stimulus output of the stimulus output module (202).

In at least an embodiment of the response intake module (204), there is provided a physiological intake module (204b) configured to sense, via one or more physiological sensors, one or physiological response signals from a user in response to the output stimulus of the stimulus output module (202). The physiological response signals may comprise physiological vectors correlative to the physiological signals in response to the stimulus output of the stimulus output module (202). These vectors are parsed through a fourth vector engine (238) configured to determine a third state of a user in correlation to such stimulus vectors of the stimulus output of the stimulus output module (202).

In at least an embodiment of the response intake module (204), there is provided a neurological intake module configured to sense, via one or more neurological sensors, one or neurological response signals from a user in response to the output stimulus of the stimulus output module (202). The neurological response signals may comprise neurological vectors correlative to the neurological signals in response to the stimulus output of the stimulus output module (202). These vectors are parsed through a fifth vector engine configured to determine a fourth state of a user in correlation to such stimulus vectors of the stimulus output of the stimulus output module (202).

Embodiments, of the vector engines form a vocal biomarker identification engine. Typically, the vocal biomarker identification engine uses a 3-step approach.
Domain knowledge to construct voice data collection protocols to enhance presence and stability of vocal biomarkers associated with depression;
Identify the best set of features capturing the subtle acoustic differences in depressed and non-depressed individuals; these features need to have good detection capabilities and robust to noise-thus, they can be used in detecting depression in natural environments;
Vocal biomarkers appearing in speech systems is a non-binary scenario; therefore, for a person suffering from depression requires a correct task and a pre-determined time of executing the task for the vocal biomarkers to have a visibility or presence when using an automatic detection system.

This detection time or Task Based Detection Threshold (TBDT) may vary with respect to gender, depression severity, and age. Therefore, there is need to conduct a procedure to identify detection threshold/s and a Detection Confidence Metric Score (DCMS) by arranging the Voice Tasks by an efficient order in such way that the system and method, of this invention, can have a stable detection range for symptoms.

Embodiments, of the present invention, may include a protocol construction engine.

Automatic speech-based processing using machine learning is being used more in digital healthcare and has the potential to be a non-invasive, remote medical screening tool. However, there is a need for more understanding of the protocols used to process speech and for measurements to help create new protocols with specific criteria.

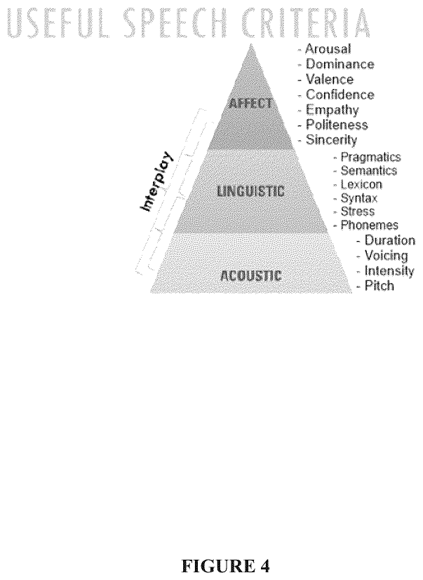

FIG. 4 illustrates known interplay between components, of speech, forming useful speech criteria.

Healthcare clinicians use speech-language evaluations to screen, diagnose, and monitor various disorders. During evaluations, clinicians observe a patient's speech production, including articulation, breathing, phonation, and voice quality, as well as the patient's language ability, such as grammar, pragmatics, memory, and expressive capacity. Abnormal speech and language symptoms can often be early indicators of different disorders and illnesses.

In at least an embodiment of the response intake module (204), there is provided a passage reading module configured to allow users to read passage basis pre-defined tasks/prompts. Advantages of read speech protocols include ease of use, repeatability, and ability to provide a clear reference point, as well as having a limited range of sounds used and controlled variations. Additionally, they are relatively simple to integrate into digital smart device applications. When choosing a read speech protocol for analyzing medical conditions, factors such as the background of the speaker, the specific illness being focused on, and the amount of time and number of samples needed are important to consider.

In at least an embodiment of the passage reading module, there is provided an emotion based fixed reading passages example.

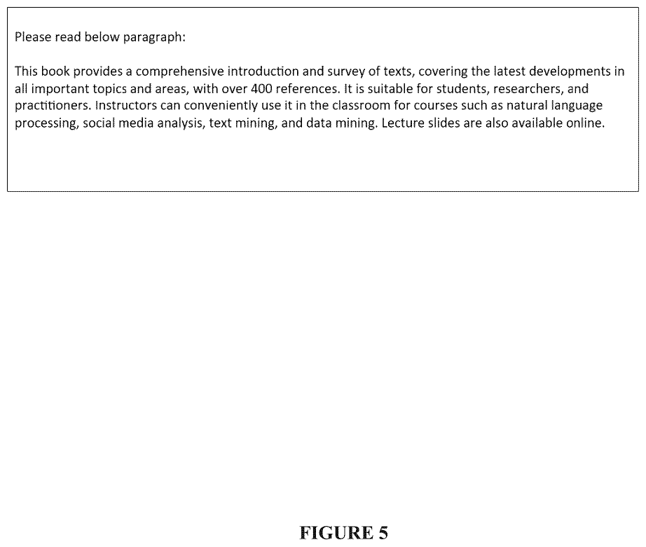

FIG. 5 illustrates one such fixed reading passage.

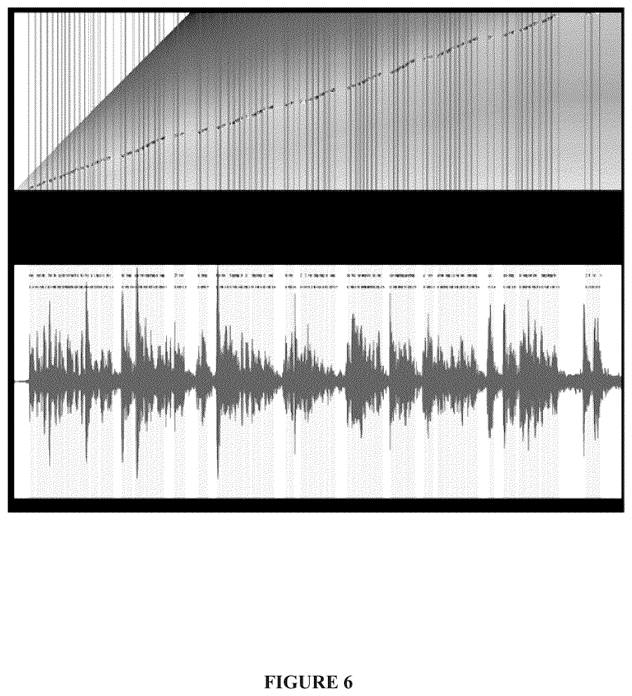

FIG. 6 illustrates a phenome map, basis read passage by a user, without tones.

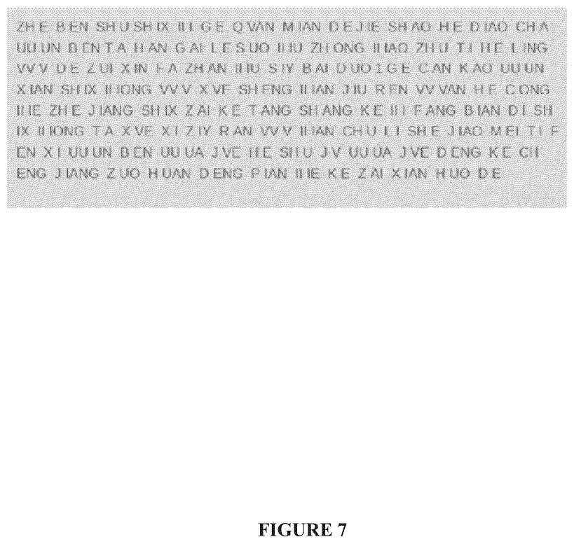

FIG. 7 illustrates another such fixed reading passage.

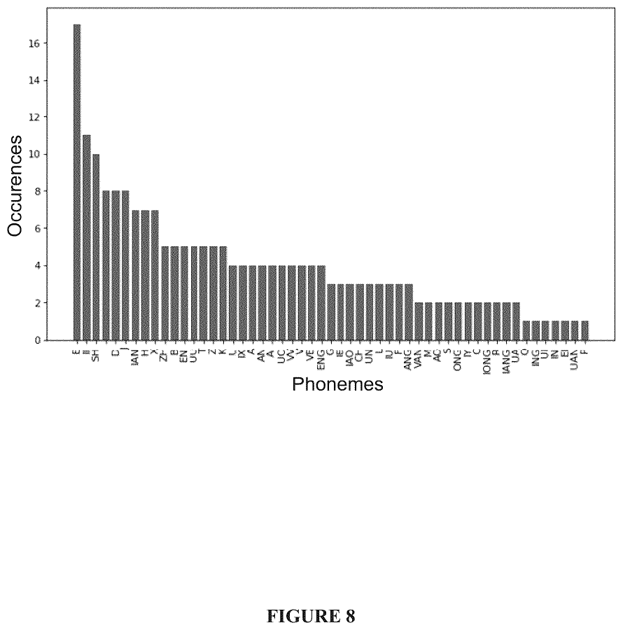

FIG. 8 illustrates a graph of phenomes versus occurrence, basis read passage by a user.

Embodiments, of the present disclosure, may include a features' module with a feature constructor to define features, for each of the constructed task, the features being defined in terms of learnable heuristic weighted tasks.

In at least an embodiment of the feature constructor, there is used a Geneva Minimalistic Acoustic Parameter Set (GeMAPS). The Geneva Minimalistic Standard Parameter Set is a set of 62 parameters used to analyze speech. It uses a symmetric moving average filter 3 frames long to smooth over time, with the smoothing only performed within voiced regions for pitch, jitter, and shimmer. Arithmetic mean and coefficient of variation are applied as functionals to all 18 LLD, yielding 36 parameters. Additionally, 8 functionals are applied to loudness and pitch, and the arithmetic mean of the Alpha Ratio, the Hammarberg Index, and the spectral slopes from 0-500 Hz and 500-1500 Hz over all unvoiced segments are included. Temporal features, such as the rate of loudness peaks, the mean length and standard deviation of continuously voiced and unvoiced regions, and the number of continuous voiced regions per second, are also included. No minimal length is imposed on voiced or unvoiced regions, and the Viterbi-based smoothing of the F0 contour prevents single voiced frames which are missing by error.

eGeMAPS (Geneva Minimalistic Acoustic Parameter Set) is a set of features used in openSMILE (The Munich Open-Source Multimodal Interface for Language and Emotion Recognition) for the analysis of speech, audio, and music. eGeMAPS is a subset of the larger Geneva Minimalistic Acoustic Parameter Set (GeMAPS) and it is specifically designed for the task of emotion recognition. It includes a set of low-level descriptors (LLDs) for the analysis of the spectral, pitch, and temporal properties of the audio signal. The features include:
Mel-Frequency Cepstral Coefficients (MFCCs) and their first and second derivatives
Pitch and pitch variability
Energy and energy entropy
Spectral centroid, spread and flatness.
Spectral slope
Spectral roll-off.
Spectral variation.
Zero-crossing rate
Shimmer, jitter and harmonic-to-noise ratio.
Voice-probability (based on pitch)
Temporal features like the rate of loudness peaks, and the mean length and standard deviation of continuously voiced and unvoiced regions

In total, it contains 87 features

eGeMAPS is designed to be a minimal set of features that are robust to different recording conditions and speaker characteristics, and it has been shown to be effective in several emotion recognition tasks.

### Frequency Related Parameters:

Pitch, logarithmic F0 on a semitone frequency scale, starting at 27.5 Hz (semitone 0)
Jitter, deviations in individual consecutive F0 period lengths.
Formant 1, 2, and 3 frequency, centre frequency of first, second, and third formant.
Formant 1, bandwidth of first formant
Energy/Amplitude related parameters.
Shimmer, difference of the peak amplitudes of consecutive F0 periods
Loudness, estimate of perceived signal intensity from an auditory spectrum
Harmonics-to-Noise Ratio (HNR), relation of energy in harmonic components to energy in noiselike components.
Spectral (balance) parameters
Alpha Ratio, ratio of the summed energy from 50-1000 Hz and 1-5 kHz
Hammarberg Index, ratio of the strongest energy peak in the 0-2 kHz region to the strongest peak in the 2-5 kHz region
Spectral Slope 0-500 Hz and 500-1500 Hz, linear regression slope of the logarithmic power spectrum within the two given bands
Formant 1, 2, and 3 relative energy, as well as the ratio of the energy of the spectral harmonic peak at the first, second, third formant's centre frequency to the energy of the spectral peak at F0
Harmonic difference H1-H2, ratio of energy of the first F0 harmonic (H1) to the energy of the second F0 harmonic (H2)
Harmonic difference H1-A3, ratio of energy of the first F0 harmonic (H1) to the energy of the highest harmonic in the third formant range (A3).

In at least an embodiment of the feature constructor, there is used Higher order spectra (HOSA) functions. Higher order spectra (HOSA) are functions of two or more component frequencies, in contrast to the power spectrum, which is a function of a single frequency. These spectra can be used to identify phase coupling between Fourier components, and they are particularly useful in detecting and characterizing nonlinearity in systems. To achieve this, the magnitude of the higher order spectrum is normalized with powers at the component frequencies. The normalized higher-order spectra, also called nth-order coherency index, is a function that combines the cumulants spectrum of order n with the power spectrum.

The bispectrum is a method that uses the third-order cumulants to analyze the relation between frequency components in a signal, and it is particularly useful for examining nonlinear signals. The bispectrum is more informative than the power spectrum, as it provides information about the phase relationships between frequency components, which is not presented by the spectral domain. Higher Order Statistics is an effective method for studying nonlinear signals, as it encloses the relations between phase components. The bispectrum is one of the best methods for this purpose, as it shows information that is not presented by the spectral domain.

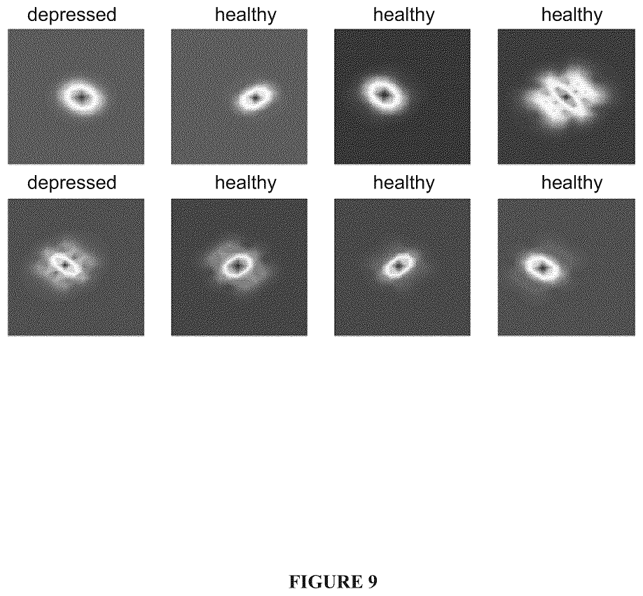

FIG. 9 illustrates representations of HOSA functions with respect to various mental states of a user.

Embodiments, of the present disclosure, may include a features' module with a feature extractor with a variable/heuristic weighted model considering at least one selected from a group consisting of ranked tasks, ranked questions, and the like. In preferred embodiments, weights per question/per task are different and are learnable in nature. Preferably, the feature extractors fuse higher-level feature embeddings using mid-level fusion.

Embodiments, of the feature extractors, are discussed in this paragraph. When training the tasks (e.g. acoustic questions), they may be assigned a dedicated feature extractor with learnable weights per stimulus (e.g. question).

In preferred embodiments, linguistic tasks/stimulus (questions) may be assigned a dedicated linguistic feature extractor with learnable weights per stimulus (e.g. question).

In preferred embodiments, affective tasks/stimulus (questions) may be assigned a dedicated affective feature extractor with learnable weights per stimulus (e.g. question).

In preferred embodiments, a selector is configured to select a random sample, of response vectors, basis pre-set thresholds of signal-to-noise-ratio, response (voice) clarity, and the like.

Embodiments, of the present disclosure, may include a data loader configured to, randomly, upsample or downsample response vectors until a balanced data batch, for training, is achieved. When constructing the data loader randomly upsample or downsample accordingly until there is achieved a balanced data batch for a training. Embodiments may also include-Due to ordinal nature of the data, the system and method is configured to use soft labels instead of hard labels for classification problem.

Despite attempts to utilize unimodal emotion feature representations, prior arts have proven insufficient in achieving recognition due to a lack of distinctiveness and an inability to effectively capture the dynamic interplay between emotions in speech recognition tasks. Therefore, embodiments, of the present disclosure, may include a features' module with a feature fusion module configured towards fusing the aforementioned various output stimuli and/or states of the user. Typically, the feature fusion modules works in consonance with the response intake module (204).

In preferred embodiments, the feature fusion modules comprise an audio modality configured with high-level feature extractors and at least an autoencoder-based feature fusion to classify mental health state from the plurality of types of response vectors. In preferred embodiments, the audio modality uses a local question-specific feature extractor to extract high-level features from a time-frequency domain relationship in the response vectors. Output, here, is extracted high-level audio features.

In preferred embodiments, the feature fusion modules comprise a text modality configured with high-level feature extractors and at least an autoencoder-based feature fusion to classify emotions from the plurality of types of response vectors. In preferred embodiments, the text modality uses a Bidirectional Long Short-Term Memory network with an attention mechanism to simulate an intra-modal dynamic. Output, here, is extracted high-level text features.

In preferred embodiments, of the audio modality, of the feature fusion modules, the system and method, uses features in acoustic feature embeddings from pre-trained models such as huBert, Wav2vec, Whisper where a raw audio input is used, highlevel embeddings from pre-trained models are extracted which are high dimensional vector embeddings. Similarly, the system and method, of this invention, extracts acoustic feature embeddings (from GeMAPS, emobase egemap), compare on spectral domain the system and method extracts log mell spectrograms and MFCC as well as Higher order spectral features (HOSA) in addition psychomotor retardation and neuro muscular activation features are extracted using vocal tract coordination features and recurrent quantification analysis features. Bigram counts and bigram durations are calculated with speech landmarks for articulation efficacy. All the features are fused together with an auto encorder where non-linearity of the features are persisted while fusing the features in the latent feature space.

Previous research has shown that the use of lexical information can improve valence estimation performance. Lexical information can be obtained from pre-trained acoustic models, where the learned representations can improve valence estimation from speech. The system and method, of this invention, investigates use of pre-trained model representations as well as task specific feature extractors such as neuro muscular coordination features estimating psychomotor retardation to improve depression biomarker estimation from acoustic speech signal. The system and method, of this invention, also explore fusion of representations to improve depression biomarker estimation. Human speech communication broadly consists of two layers the linguistic layer, which conveys messages in the form of words and their meaning, and the paralinguistic layer, which conveys how those words have been the, including vocal expressiveness or emotional tone.

It is hypothesized that given the self-supervised learning architecture of the pretrained models and the large speech data-sets they were exposed to, representations generated by these pre-trained models may contain lexical information which facilitate better valence estimation

In some embodiments, the system and method, of this invention, explore a multi modal-granularity framework to extract speech embeddings from multiple levels of subwords. As illustrated the embeddings pertained models extracted are normally frame-level embeddings and it has shown to be effective in obtaining abundant frame level information. However, it lacks the ability to capture segment-level information which is useful for depression biomarker recognition. Thus, in addition to frame-level embeddings, the system and method, of this invention, introduce segment-level embeddings including word, phone and syllabl level embeddings which are closely related to the prosody. Prosody can convey characteristics of the utterance like depression state because it contains the information of the cadence of speech signals. As a result, segment-level embeddings may be helpful for multimodal depression biomarker recognition.

Using force alignment approaches, the temporal boundaries of phonemes can be
obtained, which can then be grouped to get the boundaries of the syllables. Force
alignment information is provided and related speech segments corresponding to those units can be extracted thereafter.

Embodiments, of the present disclosure, may include a speech landmark extractor.

Speech landmarks are event markers that are associated with the articulation of speech. They rely solely on the location of acoustic events in time, such as consonant closures/releases, nasal closures/releases, glide minima, and vowel maxima, providing information about articulatory events such as the vibration of the vocal folds. Unlike the frame-based processing framework, landmark methods detect timestamp boundaries that denote sharp changes in speech articulation, independently of frames. This approach offers an alternative to frame-based processing, and has the potential to circumvent its drawbacks, by focusing on acoustically measurable changes in speech. VTH adopts six landmarks, each with onset and offset states. They are ‘g(lottis)’, ‘p(eriodicity)’, ‘s(onorant)’, ‘f(ricative)’, ‘v(oiced fricative)’, and ‘b(ursts)’, which are used to specify points in time at which different abrupt articulatory events occur. They are detected by observing evidence of rapid changes (i.e. rises or falls) in power across multiple frequency ranges and multiple time scales. Among the landmarks, ‘s’ and ‘v’ relate to voiced speech, while ‘f’ and ‘b’ relate to unvoiced speech.

Embodiments, of the present disclosure, may include a vocal track coordination engine

VTC features have demonstrated the ability to capture depression-related psychomotor activity, were most successful at two A VEC challenges on depression severity prediction, and are predicated on the observation that vocal tract parameters are less ‘coordinated’ (correlated) for depressed than for healthy speakers.

TABLE 2

| Landmark | Description |
|---|---|
| g | sustained vibration of vocal folds starts (+) or |
| | ends (−). |
| p | sustained periodicity begins (+) or ends (−) |
| s | releases (+) or closures (−) of a nasal |
| f | frication onset (+) or offset (−) |
| v | voiced frication onset (+) or offset (−) |
| b | onset (+) or offset (−) of existence of turbulent |
| | noise during obstruent regions |

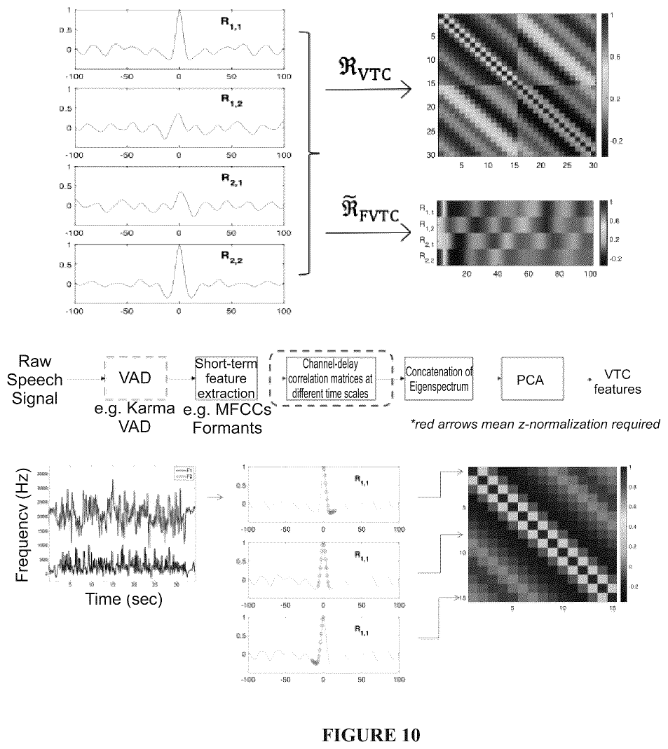

FIG. 10 illustrates autocorrelations and cross-correlations between the 1st and the 2nd delta MFCCs extracted from a 10-s speech file, framework for extracting the delayed correlations from acoustic files reflecting the psychomotor retardation.

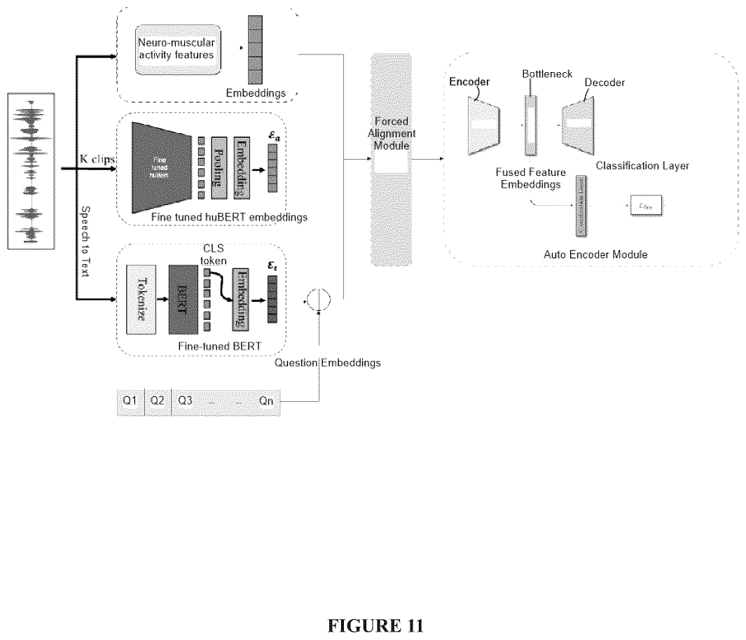

FIG. 11 illustrates a schematic block diagram of an autoencoder.

Embodiments, of the present disclosure, may include an autoencoder to define relationship/s between the audio modality (of the feature fusion module working in consonance with the response intake module (204)) and the text modality (of the feature fusion module working in consonance with the response intake module (204)) in consonance with the stimulus output module (202). The autoencoder is configured such that once the aforementioned processing, vide audio modality and text modality is completed, the extracted high-level text features and the extracted high-level audio features may be fed into an autoencoder, in parallel, to output a shared representation feature data set for emotion classification. This is unique since the system and method, of this invention, not only measures accuracy of the autoencoder in reconstructing the shared representation feature data set and minimizes reconstruction error/s, but also evaluate performance of depression detection in the emotion recognition module (300).

This is different from prior systems and methods that only focus on learning high-level features from input data using autoencoders. Autoencoders are excellent at capturing low-level shared representations, in a non-linear manner; this strength is utilized in the current invention's system and method so that closed fixed tasks (such as reading paragraphs, counting or phoneme articulation tasks) are evaluated with the best local feature extractors while the open-ended tasks (questions) are evaluated with both, acoustic and text, modalities while optimizing losses.

An autoencoder is a type of neural network that can be used for feature fusion for audio data. It can be used for multi-modal feature fusion by combining the data from multiple modalities (e.g. audio representations and text representations) into a single joint representation. This can be done by training an autoencoder to encode the input data into a lower-dimensional space, and then decode it back to the original space. The encoded representation is the bottleneck or the compact feature representation that captures the most important information from the input data.

High-level overview an autoencoder architecture used for multi-modal feature fusion for audio data:
1. Collect and preprocess the audio data and other modality data.
2. Build an autoencoder architecture with an encoder and a decoder.
3. The encoder takes the input data from both audio and other modality and compresses it into a lower-dimensional representation (bottleneck or latent representation).
4. The decoder takes the bottleneck representation and reconstructs the original input data.
5. Train the autoencoder by providing the input data from both modalities as input to the encoder and the original input data as the target output for the decoder.
6. After training, the bottleneck representation is used as the fused feature representation for both modalities.
7. This fused feature representation can be used for further analysis, such as classification or clustering.

Intermediate fusion strategies utilize prior knowledge to learn marginal representations

of each modality, discover within-modality correlations, and then use these to either learn joint representations or make predictions directly.

Intermediate fusion strategies can mitigate an imbalance in dimensionality between modalities by forcing the marginal representations to be of similar size, but if the imbalance is very large, reducing the dimensionality of the larger modality too much might result in significant loss of information. However, if the input features of the lower dimensional modality are chosen with prior knowledge, imbalance does not necessarily lead to poor performance.

Each speech task in the current invention's dataset contributes to a very specific emotion derivative for example the words in the paragraph 1 consist of positive majority emotion words which are couples with neutral sentiment resulting readers to have positive intention while utterance for healthy individuals, once words with in the paragraph or positive negative and neutral paragraphs itself while utterance captures the affective information related to the acoustic information which is an indicator for detecting the phoneme/affective information differences between healthy and patients.

In-order to use all the different questions in the same model, the inventors assign the questions a question embedding which is a 0-N vector where the question specific feature extractors will have prior knowledge on which word, phone and syllabl-level embeddings need to be extracted from the data and forced aligned for the mid-level feature fusion.

The prior knowledge comes from linguist and phonetic experts about specific tasks and order of the tasks verified from the model evaluation process developing integrated gradients using SHAP values for raw audio signals.

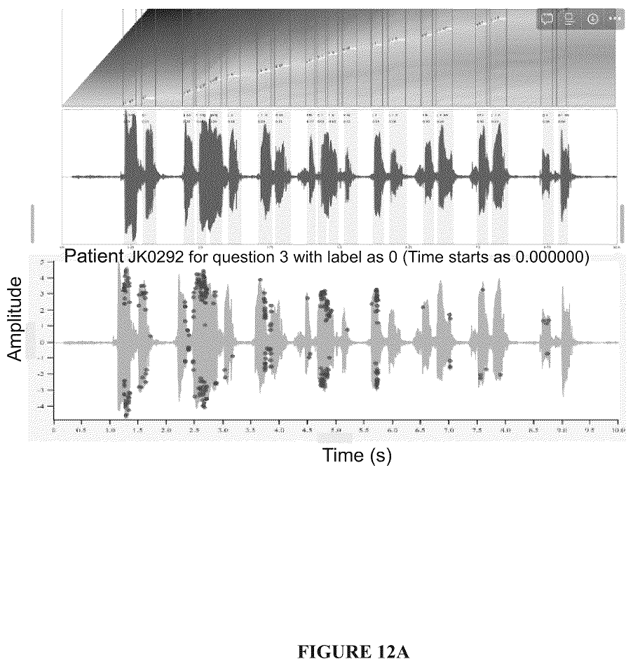

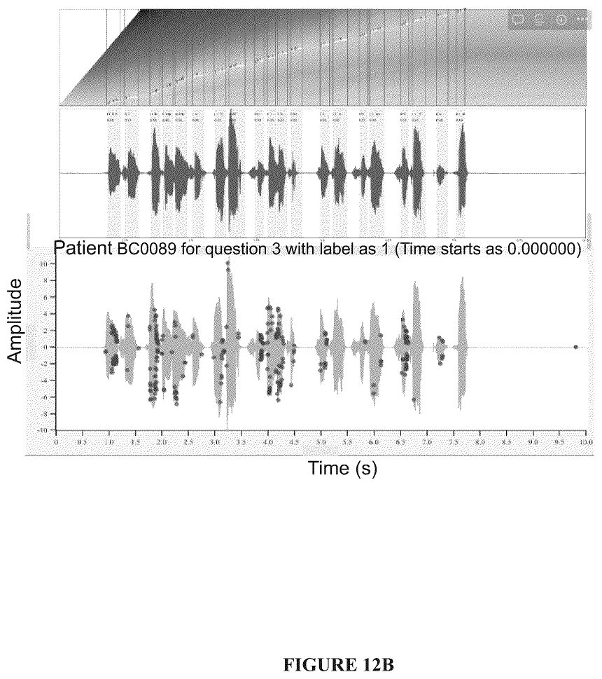

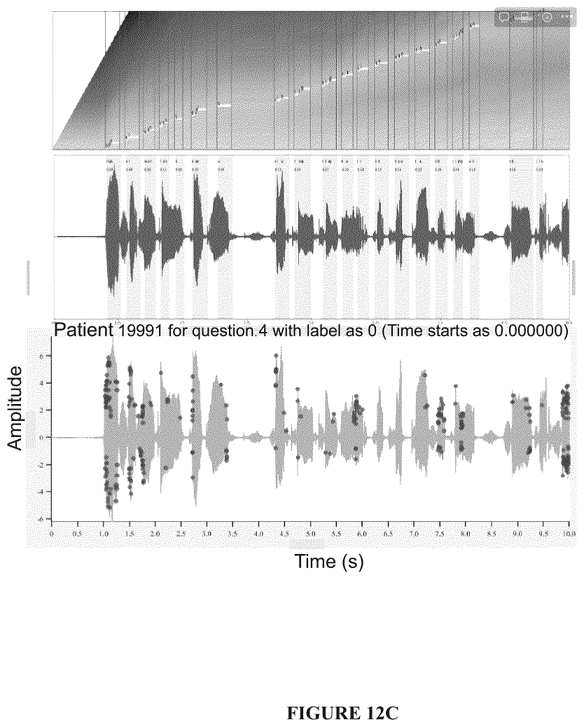

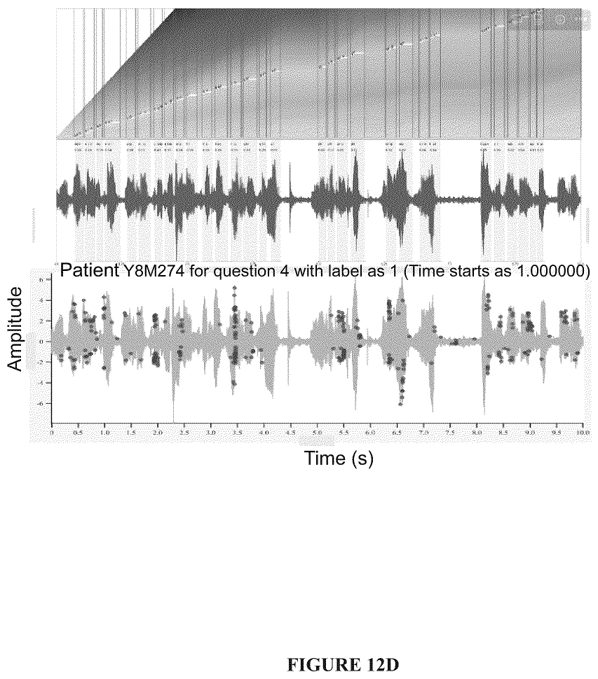

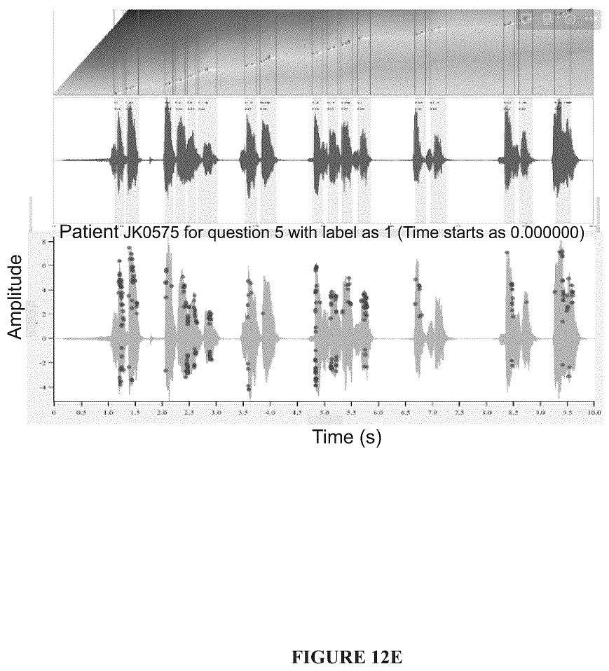

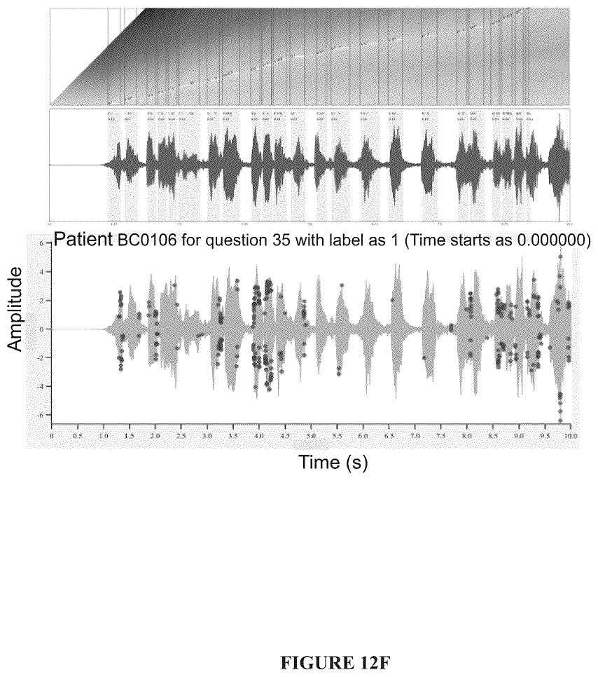

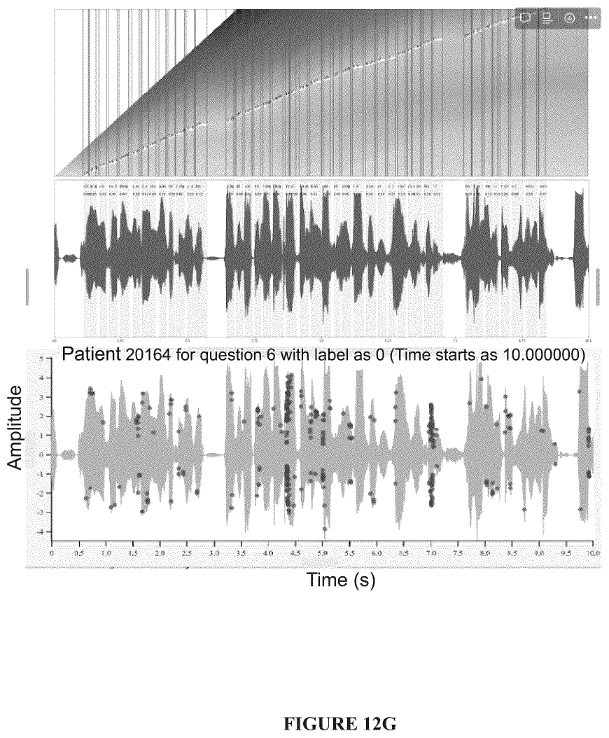

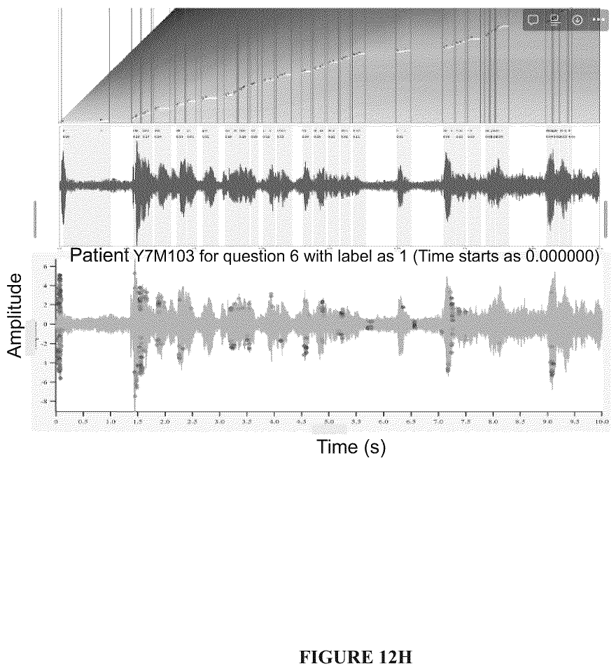

FIG. 12A illustrates a graph, for at least one type of task (question), according to a non-limiting exemplary embodiment, with its original label being ‘healthy’, the graph showing the phonemes and segments that have positive correlations with the model prediction, the areas highlight the parts of speech file that have features with high activation.

FIG. 12B illustrates a graph, for at least one type of task (question), according to a non-limiting exemplary embodiment, with its original label being ‘depressed’, the graph showing the phonemes and segments that have positive correlations with the model prediction, the areas highlight the parts of speech file that have features with high activation for a second user (at start time 0 s).

FIG. 12C illustrates a graph, for at least one type of task (question), according to a non-limiting exemplary embodiment, with its original label being ‘healthy’, the graph the phonemes and segments that have positive correlations with the model prediction, the areas highlight the parts of speech file that have features with high activation for a third user (at start time 0 s).

FIG. 12D illustrates a graph, for at least one type of task (question), according to a non-limiting exemplary embodiment, with its original label being ‘depressed’, the graph showing the phonemes and segments that have positive correlations with the model prediction, the areas highlight the parts of speech file that have features with high activation for a fourth user (at start time 1 s).

FIG. 12E illustrates a graph, for at least one type of task (question), according to a non-limiting exemplary embodiment, with its original label being ‘healthy’, the graph showing the phonemes and segments that have positive correlations with the model prediction, the areas highlight the parts of speech file that have features with high activation for a fifth user (at start time 0 s).

FIG. 12F illustrates a graph, for at least one type of task (question), according to a non-limiting exemplary embodiment, with its original label being ‘depressed’, the graph showing the phonemes and segments that have positive correlations with the model prediction, the areas highlight the parts of speech file that have features with high activation for a sixth user (at start time 0 s).

FIG. 12G illustrates a graph, for at least one type of task (question), according to a non-limiting exemplary embodiment, with its original label being ‘healthy’, the graph showing the phonemes and segments that have positive correlations with the model prediction, the areas highlight the parts of speech file that have features with high activation for a seventh user (at start time 10 s).

FIG. 12H illustrates a graph, for at least one type of task (question), according to a non-limiting exemplary embodiment, with its original label being ‘depressed’, the graph showing the phonemes and segments that have positive correlations with the model prediction, the areas highlight the parts of speech file that have features with high activation for an eighth user (at start time 0 s).

The mathematical equation for an autoencoder-based multi-modal multi-question input fusion architecture can be represented as:
Let X1, X2, X3, . . . , Xn be the n modalities (e.g. audio, text) and Q1, Q2, Q3, . . . , Qm be the m questions. each question will be multiplied with a learnable weight a encoded matrix based on the question type.

The encoder function E maps the n modalities and m questions to a lower-dimensional representation Z:

Z=E(X1,X2,X3, . . . ,Xn,Q1,Q2,Q3, . . . ,Qm)

The decoder function D maps the lower-dimensional representation Z back to the original input space:

X′1,X′2,X′3, . . . ,X′n=D(Z)

The autoencoder is trained to minimize the reconstruction error between the original input and the decoder output, typically using a loss function such as mean squared error (MSE) or cross-entropy:

L=1/nmΣ(X′i−Xi)\hat{ }2 or L=−1/nmΣXi*log(X′i)

The lower-dimensional representation Z is the fused feature representation of the n
modalities and m questions, which can be used for further analysis, such as
classification or clustering, the encorder weights will be used as a feature extractor for the downstream task of depression classification.

An autoencoder based feature fusion is obtained as follows. First, the hidden representations for audio input and the respective text input is obtained using HuBERT and BERT models and the psychomotor retardation features, respectively. Since the dataset contains similar answers for a significant number of questions, a weight representing the question id is also fed into the overall model. The question id weight is used to multiply the text hidden features, element-wise. The resulting text and audio embeddings are then concatenated with forced alignment and such concatenated features are used to train an autoencoder model to obtain a fused representation from the bottleneck layer. The learned autoencoder weights until the bottleneck layer are then loaded to a model, and a classier head is attached to the bottleneck layer to train a classification model.

In some embodiment, subjects (users), in response to the data from the stimulus output module (202), through their client devices (112, 114, 116, 118), interacts with the response intake module (204) to record their responses.

In at least an embodiment, the various vector engines (232, 234, 236, 238) are configured to be a part of the networked server (100) and are, further, communicably coupled to an emotion recognition module (300) and to an automatic speech recognition module (400).

In preferred embodiments, the responses, at the client devices (112, 114, 116, 118), are sampled at a sampling rate of 16 KHz and, then, sent back to the networked server (100) from where it is sent to the emotion recognition module (300). The emotion recognition module (300) produces a multi-class emotion, via one or more emotion vectors, interleaved with the one more stimuli, together with their confidence. If the emotion does not align with the target induced emotion with a prefigured confidence, the user will get prompted to answer again. In some embodiments, a first pre-defined stimulus with a first pre-defined emotion vector is presented to a user, via the stimulus output module (202) in order to record responses, and correlative response vectors, through the response intake module (204). In some embodiments, a first pre-defined stimulus with a second pre-defined emotion vector is presented to a user, via the stimulus output module (202) in order to record responses, and correlative response vectors, through the response intake module (204). Thus, a particular pre-defined stimulus may be configured with one or more pre-defined emotion vectors and a variety of combinations of stimulus and emotion vectors can be presented to a user, via the stimulus output module (202) in order to record responses, and correlative response vectors, through the response intake module (204).

In preferred embodiments, the responses, at the client devices (112, 114, 116, 118), are sampled at a sampling rate of 16 KHz and, then, sent back to the networked server (100) from where it is sent to the automatic speech recognition module (400). The response data are also transcribed with automatic speech recognition technology.

In at least an embodiment, the response data, their transcription, as well as the recognized emotions are saved into a server training store/server training database (122).

In at least an embodiment, an iteration and prioritisation module is configured to prioritize stimulus based on pre-defined rules applied to incoming corresponding responses. E.g. separating different stimuli with neutral ones, happiness before sadness, and the like. If that happens, the saved data should reflect the order of the stimuli questions.

In at least an embodiment, the networked server (100) is communicably coupled with a multi-modal mental health classifier (500) (being the autoencoder) which is an Artificial Intelligence (AI) module configured to be trained to classify mental health issues based on voice recordings as well as on textual responses.

Artificial intelligence (“AI”) module may be trained on structured and/or unstructured datasets of stimuli and responses. The training may be supervised, unsupervised or a combination of both. Machine learning (ML) and AI algorithms may be used to learn from the various modules. The AI module may query the vector engines to identify response to stimuli, order of stimuli, latency of responses, and the like in order to learn rules regarding the state of a user's mental health (defined by confidence scores). Deep learning models, neural networks, deep belief networks, decision trees, genetic algorithms as well as other ML and AI models may be used alone or in combination to learn from the solution grids.

Artificial intelligence (“AI”) module may be a serial combination of CNN and LSTM.

A high-level representation of a raw waveform and short-term temporal variability along with long-term temporal variability is captured in order to accurately build a relationship for vocal biomarkers and language models specific for users (subjects) suffering from a specific (depression) mental health condition. Considering the deep features comparing to shallow features the model encodes the depression-related temporal clues which are present when the motor cortex is affected.

As mentioned in the data collection part, data was collected with different stimuli question order. The above training process was repeated on this specialized data set as well to ensure that the performance gain from the induced emotion was not affected by leading questions.

Given the current data size, nothing obvious was noticeable. With more and more data collected using different orders and combinations, the system and method, of this invention, might have new findings one day. And when that happens, it's easy to compare results from these different “experiments” and pick up the best stimuli order.

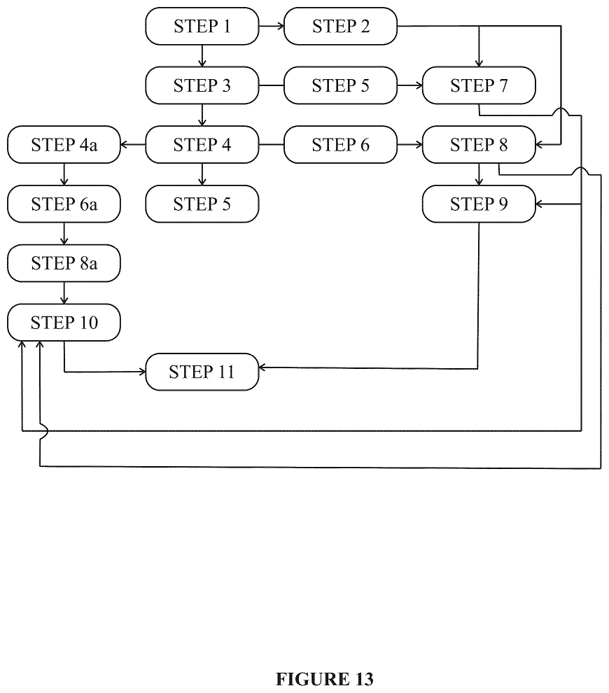

FIG. 13 illustrates a flowchart.

STEP 1: Present a user with a stimulus to elicit an acoustic response.

STEP 2: Store vectors, relating to emotion, in relation to various stimuli.

STEP 3: Record the user's acoustic response.

STEP 4: Transcribe, and record, a text version of the user's recorded acoustic response.

STEP 4a: Optionally, record the user's physiological response.

STEP 5: Extract, and analyse, vectors from the user's acoustic response, for emotion recognition (obtained through emotion detection model which produces multi-class emotion) classifications in terms of emotion-recognised information.

STEP 6: Extract, and analyse, vectors from the user's textual response, for emotion recognition in terms of emotion-recognised information.

STEP 6a: Optionally, Extract, and analyse, vectors from the user's physiological response, for emotion recognition (obtained through emotion detection model which produces multi-class emotion) in terms of emotion-recognised information.

STEP 7: Compare emotion-recognised information, from textual response, with emotion signal vectors from a training dataset, in correlation with vectors, relating to emotion, in relation to various stimuli, to obtain a first spatial vector distance and a first weighted vector difference.

STEP 7a.1: Optionally, analyze training data set for certain stimuli, first, and get top emotions associated with answers from patients for that stimulus, as well as the confidence score threshold associated by using a preconfigured percentile.

STEP 7a.2: Record those emotions and confidence score threshold associated with the given stimuli to a database.

STEP 7a.3: During runtime, for each input, get the emotion classes/confidence scores, and then compare with the data recorded in STEP 7a.2.

STEP 8: Compare emotion-recognised information, from acoustic response, with emotion signal vectors from a training dataset, in correlation with vectors, relating to emotion, in relation to various stimuli, to obtain a second spatial vector distance and a second weighted vector difference.

STEP 8a: Optionally, compare emotion-recognised information, from physiological response, with emotion signal vectors from a training dataset, in correlation with vectors, relating to emotion, in relation to various stimuli, to obtain a third spatial vector distance and a third weighted vector difference.

STEP 8b: Optionally, as an alternative to STEP 7 and STEP 8, obtain an emotion output using emotion recognized information from different modalities (acoustic, and/or with textual, and/or with physical) by using a fusion layer first, then compare it with emotion signal vectors.

STEP 9: Conflate, intelligently, the first spatial vector distance, the first weighted vector difference, the second spatial vector distance, the second weighted vector difference to obtain a first confidence score.

STEP 10: Optionally, Conflate, intelligently, the first spatial vector distance, the first weighted vector difference, the second spatial vector distance, the second weighted vector difference, the third spatial vector distance, the third weighted vector difference to obtain a second confidence score.

STEP 11: Iterate stimulus (STEP 1), to obtain a further acoustic response, and correlative transcribed textual response, based on recognised emotion, with respect to the first confidence score and/or the second confidence score.

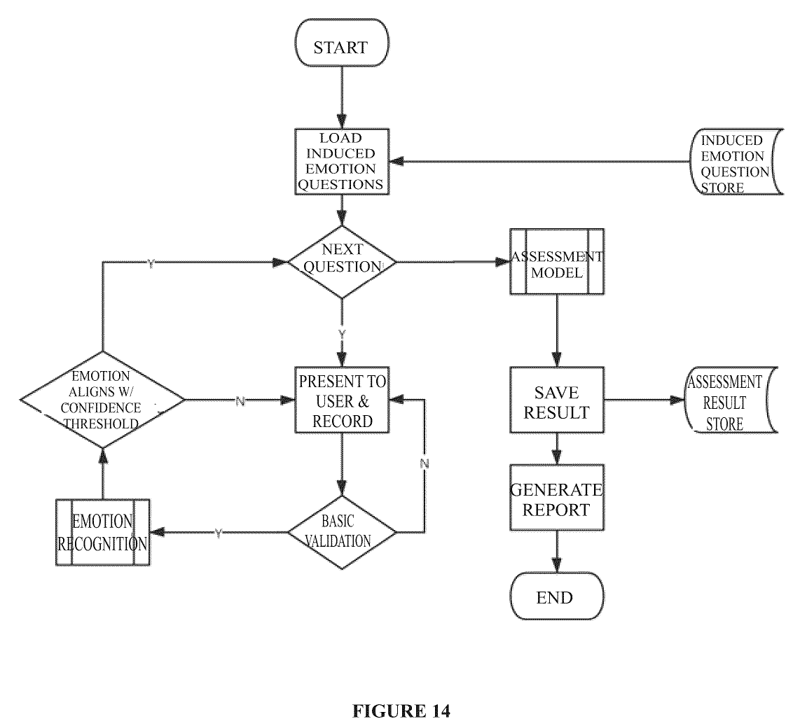

FIG. 14 illustrates the high-level flowchart for voice-based mental health assessment with emotion stimulation.

According to a non-limiting exemplary embodiment, in order to capture effects of induced emotions (stimuli vide multimedia output stimulus) to the invented system's performance, a model was trained for each induced emotion leveraging a given data set. A comparison was done in order to compare the performance of various such models (can be done either leveraging n-fold cross validation or on a dedicated validation set), and the top three questions (stimuli) with the best performance were picked. A possible alternative approach is to train one single model on all the data combined, and then run the model against validation sets consisting of answers for one single stimulus. A single measurement (e.g. AUROC) is then used to compare model performance for each stimulus, and the top three stimuli are selected.

Users provide responses to such stimuli, one by one, in their own environment using their respective client devices. For each response received, the system and method, of this invention, does some preprocessing and validation (e.g. voice activation, loose length check, and the like) and, then, runs it through the emotion recognition model (200). If the recognized emotion and its confidence score aligns with the induced emotion, the system and method, of this invention, moves on to the next question. Otherwise, the system and method, of this invention, will induce stimulus in order to prompt the user to invoke response to the same stimulus, again. This process continues. If the user's response attempt exceeds certain criteria for the same stimulus, but the emotion recognition still fails, a similar substitute stimulus, under the same induced emotion, will be asked instead. And, if the total attempt exceeds a certain threshold, the system and method, of this invention, may proceed to assessment with a warning to the user that the data collected is of lower confidence.

The collected recordings, of responses and corresponding stimuli, are sent to the multi-modal mental health classifier (500), which assesses current user's mental health issue together with a correlative risk index. In preferred embodiment, the classification result for the open-ended sadness induced emotion takes precedence and is given higher weight in final result computation, and an assessment report is built and sent back to the user, as well as the provider, either via real time voice or text (through the client device), or non-real time (e.g. via email, messages etc.).

Results for all induced emotions (responses to stimuli) are recorded in an assessment result store/assessment result database (124).

TABLE 2, below, illustrates a model performance metrics (10-fold average) on top induced emotion questions. Leveraging induced emotions and emotion recognition, the system and method, of this invention, outperforms other multi-modal technologies, and brings at least 10% improvement compared to models with hand engineered features.

TABLE 2

| METRICS | VALUE |
|---|---|
| Sensitivity | 0.814 |
| Specificity | 0.866 |
| Marcro-F1 | 0.84 |
| F1 - Depressed | 0.81 |
| AUROC | 0.902 |

The TECHNICAL ADVANTAGE, of this invention, lies in providing a voice-based mental health assessment, with emotion stimulation, and monitoring system which provides much better sensitivity/specificity, by leverages induced emotions as well as emotion recognition when processing audio input from the user.

The TECHNICAL ADVANCEMENT, of this invention, lies in providing a multi-modal architecture, for voice-based mental health assessment system and method, with emotion stimulation, involving acoustic features along with speech/language features.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A multi-modal system for voice-based mental health assessment with emotion stimulation, the system comprising:
a task construction module configured to construct tasks for capturing acoustic, linguistic, and affective characteristics of speech of a user;
a stimulus output module configured to receive data from the task construction module, wherein the stimulus output module includes one or more stimuli and a basis for the constructed tasks to be presented to a user to elicit a trigger of one or more types of user behavior, wherein the triggers are in the form of input responses;
a response intake module configured to present, to the user, the one or more stimuli and the basis for the constructed tasks, from the stimulus output module, and, in response, receive corresponding responses in one or more formats;
a feature module comprising,
a feature constructor configured to define features, for each constructed task, wherein the features are defined in terms of learnable heuristic weighted tasks,
a feature extractor configured to extract one or more defined features from the received corresponding responses correlative to the constructed tasks using a learnable heuristic weighted model considering at least one task selected from a group of ranked constructed tasks,
a feature fusion module configured to fuse two or more defined features to obtain fused features; and
an autoencoder configured to define a relationship, using the fused features, between:
an audio modality of the feature fusion module working in consonance with the response intake module to extract high-level features, in the responses, and output extracted high-level text features, and
a text modality of the feature fusion module working in consonance with the response intake module to extract high-level features, in the responses, and output extracted high-level audio features, wherein the autoencoder is configured to receive extracted high-level text features and extracted high-level audio features, in parallel, from the audio modality and the text modality to output a shared representation feature data set for emotion classification correlative to the mental health assessment.

2. The system of claim 1, wherein the constructed tasks are articulation tasks and/or written tasks.

3. The system of claim 1, wherein the task construction module includes a first order ranking module configured to rank the constructed tasks in order of difficulty to assign a first order of weights to each constructed task.

4. The system of claim 1, wherein the task construction module includes a first order ranking module configured to rank the constructed tasks in order of difficulty to assign a first order of weights to each constructed task, wherein the construed tasks are stimuli marked with a ranked valence level, correlative to analyzed responses, selected from a group consisting of positive valence, negative valence, and neutral valence.

5. The system of claim 1, wherein the task construction module includes a second order ranking module configured to rank complexity of the constructed tasks in order of complexity to assign a second order of weights to each constructed task.

6. The system of claim 1, wherein the constructed tasks are at least one of, cognitive tasks of counting numbers for a pre-determined time duration, tasks correlating to pronouncing vowels for a pre-determined time duration, uttering words with voiced and unvoiced components for a pre-determined time duration, word reading tasks for a pre-determined time duration, paragraph reading tasks for a pre-determined time duration, tasks related to reading paragraphs with phoneme and affective complexity to open-ended questions with affective variation, and pre-determined open tasks for a for a pre-determined time duration.

7. The system of claim 1, wherein the constructed tasks include one or more questions, as stimulus, each question being assigned a question embedding with a 0-N vector such that a question-specific feature extractor is trained in relation to determination of embeddings extraction, from the question, correlative to word-embedding, phone-embedding, and syllable level embedding, the extracted embeddings being and forced aligned for a mid-level feature fusion.

8. The system of claim 1, wherein the one or more stimulus is at least one of audio stimulus, video stimulus, text stimulus, multimedia stimulus, and physiological stimulus, and wherein the one or more stimulus comprising stimulus vectors are calibrated to elicit at least one of textual response vectors, audio response vectors, video response vectors, multimedia response vectors, and physiological response vectors in response to the stimulus vectors.

9. The system of claim 1, wherein the one or more stimulus are parsed through a first vector engine configured to determine constituent vectors to determine a weighted base state in correlation to such stimulus vectors.

10. The system of claim 1, wherein the response intake module includes a passage reading module configured to allow users to perform tasks correlative to reading passages for a pre-determine time.

11. The system of claim 1, wherein the feature constructor is a Geneva Minimalistic Acoustic Parameter Set (GeMAPS) based feature constructor for analysis of audio responses, and wherein the GeMAPS is configured to,
use a set of 62 parameters to analyze speech;
provide a symmetric moving average filter, 3 frames long, to smooth over time, the smoothing being performed within voiced regions, of the responses, for pitch, jitter, and shimmer;
apply arithmetic mean and coefficient of variation as functionals to 18 low-level descriptors (LLDs), yielding 36 parameters;
apply 8 functions to loudness;
apply 8 functions to pitch;
determine arithmetic mean of Alpha Ratio;
determine a Hammarberg Index;
determine spectral features vide spectral slopes from 0-500 Hz and 500-1500 Hz over all unvoiced segments;
determine temporal features of continuously voiced and unvoiced regions from the responses; and
determine Viterbi-based smoothing of a F0 contour, thereby, preventing single voiced frames which are missing by error.

12. The system of claim 1, wherein the feature constructor is a GeMAPS based feature constructor configured with a set of low-level descriptors LLDs for analysis of the spectral, pitch, and temporal properties of the responses being audio responses, the features being at least one of,
Mel-Frequency Cepstral Coefficients (MFCCs) and their first and second derivatives,
pitch and pitch variability,
energy and energy entropy,
spectral centroid, spread, and flatness,
spectral slope,
spectral roll-off,
spectral variation,
zero-crossing rate,
shimmer, jitter; and harmonic-to-noise ratio,
voice-probability based on pitch, and
temporal features like the rate of loudness peaks, and the mean length and standard deviation of continuously voiced and unvoiced regions.

13. The system of claim 1, wherein the feature constructor is a GeMAPS based feature constructor configured with a set of frequency related parameters selected from at least one of:
pitch, logarithmic F0 on a semitone frequency scale, starting at 27.5 Hz (semitone 0);
jitter, deviations in individual consecutive F0 period lengths;
formant 1, 2, and 3 frequency, centre frequency of first, second, and third formant;
formant 1, bandwidth of first formant;
energy related parameters;
amplitude related parameters;
shimmer, difference of the peak amplitudes of consecutive F0 periods;
loudness, estimate of perceived signal intensity from an auditory spectrum;
harmonics-to-Noise Ratio (HNR), relation of energy in harmonic components to energy in noiselike components;
spectral balance parameters;
Alpha Ratio, ratio of the summed energy from 50-1000 Hz and 1-5 kHz;
Hammarberg Index, ratio of the strongest energy peak in the 0-2 kHz region to the strongest peak in the 2-5 kHz region;
Spectral Slope 0-500 Hz and 500-1500 Hz, linear regression slope of the logarithmic power spectrum within the two given bands;
Formant 1, 2, and 3 relative energy, as well as the ratio of the energy of the spectral harmonic peak at the first, second, third formant's centre frequency to the energy of the spectral peak at F0;
Harmonic difference H1-H2, ratio of energy of the first F0 harmonic (H1) to the energy of the second F0 harmonic (H2); and
Harmonic difference H1-A3, ratio of energy of the first F0 harmonic (H1) to the energy of the highest harmonic in the third formant range (A3).

14. The system of claim 1, wherein the feature constructor is GeMAPS based feature constructor using Higher order spectra (HOSA) functions, wherein the functions are at least one of,
functions of two or more component frequencies achieving bispectrum frequencies, and
functions of two or more component frequencies achieving bispectrum frequencies, in that, the bispectrum using third-order cumulants to analyze relation between frequency components in a signal correlative to the responses for examining nonlinear signals.

15. The system of claim 1, wherein the feature extractor fuses higher-level feature embeddings using mid-level fusion.

16. The system of claim 1, wherein the feature extractor includes a dedicated linguistic feature extractor, with learnable weights per stimulus, correlative to linguistic tasks.

17. The system of claim 1, wherein the feature fusion module includes an audio module configured with high-level feature extractors and at least an autoencoder-based feature fusion to classify emotions from one or more responses.

18. The system of claim 1, wherein the feature fusion module includes an audio module configured with high-level feature extractors and at least an autoencoder-based feature fusion to classify emotions from one or more responses, in that, the audio modality using a local question-specific feature extractor to extract high-level features from a time-frequency domain relationship in the responses so as to output extracted high-level audio features.

19. The system of claim 1, wherein the feature fusion module includes a text module configured with high-level feature extractors and at least an autoencoder-based feature fusion to classify emotions from one or more responses, in that, the text modality using a Bidirectional Long Short-Term Memory network with an attention mechanism to simulate an intra-modal dynamic so as to output extracted high-level text features.

20. The system of claim 1, wherein the feature fusion module includes an audio module configured to,
use extracted features in acoustic feature embeddings from pre-trained models,
compare the extracted features on a spectral domain,
determine vocal tract co-ordination features,
determine recurrent quantification analysis features,
determine Bigram count features and bigram duration features correlated to speech landmarks, and
fuse the features in an autoencoder.
